(19) United States
(12) Patent Application Publication
(10) Pub. No.: US 2025/0369146 A1
(43) Pub. Date: Dec. 4, 2025

(54) AMMONIUM SALT COMPOUND, ADDITIVE FOR ELECTROLYTIC COPPER PLATING, ELECTROLYTIC COPPER PLATING SOLUTION COMPOSITION AND ELECTROLYTIC COPPER PLATING METHOD

(71) Applicant: SAMSUNG ELECTRONICS CO., LTD., SUWON-SI (KR)

(72) Inventor: Kazuki Hirano, Suwon-si (KR)

(21) Appl. No.: 19/083,965

(22) Filed: Mar. 19, 2025

(30) Foreign Application Priority Data
May 31, 2024 (JP) 2024-089386

(51) Int. Cl. C25D 3/40; C25D 7/12

(52) U.S. Cl. CPC C25D 3/40; C25D 7/12

(57) ABSTRACT

An ammonium salt compound represented by the following Chemical Formula 1:

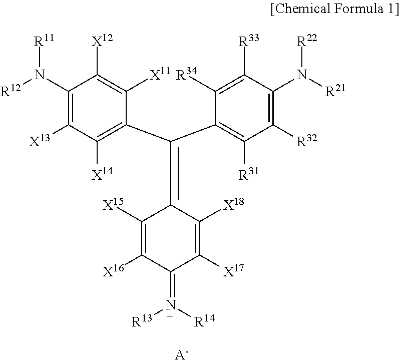

### CROSS-REFERENCE TO RELATED APPLICATION

This application claims priority under 35 U.S.C. § 119 to Japanese Patent Application No. 2024-089386, filed on May 31, 2024 in the Japanese Intellectual Property Office, the disclosure of which is incorporated by reference in its entirety herein.

### 1. TECHNICAL FIELD

The present disclosure relates to an ammonium salt compound, an additive for electrolytic copper plating, an electrolytic copper plating solution composition, and an electrolytic copper plating method.

### 2. DISCUSSION OF RELATED ART

With the recent increase in the complexity of semiconductor circuits, the technology in the plating field used to fill vias and/or trenches with high selectivity has also had increased complexity. To impart selectivity to the metal deposition on these protrusions and depressions, it is known to use organic compound called levelers as additives to the pretreatment solution for plating or as additives to the plating solution.

As a leveler used in copper plating, nitrogen-containing organic compounds are commonly used. As levelers used in copper plating, Janus Green B (JGB), Bismarck Brown Y, and Crystal Violet are known in JP2021-185271A; JP59-27992A; JP2004-360055A; JP2015-503033A and JP2018-111863A. In addition, as levelers used in copper plating, a reaction product of a nitrogen-containing organic compound and a compound having an ether structure, a compound having an alkylene oxide structure, or a compound having an epoxide structure is known in JP2004-360055A; JP2017-532452A; JP2016-155996A and JP2019-85647A. In addition, as levelers used in plating such as copper plating, rosalin, tetramethyl pararosalin hydrochloride, pentamethyl pararosalin hydrochloride, and hexamethyl pararosalin hydrochloride are known in JP59-27992A; JP2015-503033A; JP2014-505167A and US 2019/0100848A. In addition, as levelers used in plating such as copper plating, an oligomer or polymer having a biguanide structure, ionic compounds including a quaternary ammonium cation, and ionic compounds including a partial structure derived from an unsaturated heterocyclic compound including one or more heteroatoms (e.g., nitrogen atoms, etc.) are known in JP2014-505167A and JP2018-530675A.

However, with known plating solutions using a leveler, there are cases where the filling of the concave portion of the plated object by plating does not proceed sufficiently, the plating film on the concave portion of the plated film becomes dented, and it becomes difficult to form a plating film with a flat surface.

### SUMMARY

The present disclosure provides an ammonium salt compound, additive for electrolytic copper plating, electrolytic copper plating solution composition and electrolytic copper plating method.

An object of the present disclosure is to provide a means of enabling the formation of a plating film with excellent filling properties and high flatness of the concave portion of the plating object.

In the process, by using a nitrogen-containing organic compound with a specific structure for plating, the above problems can be solved.

According to an embodiment of the present disclosure, an ammonium salt compound represented by the following chemical formula 1 is provided.

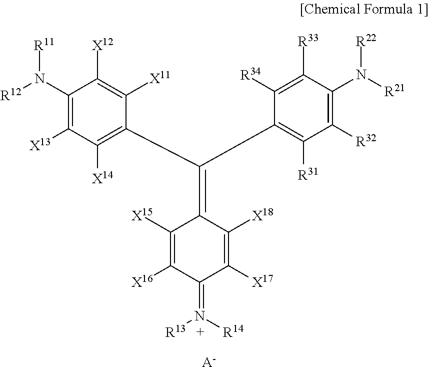

In Chemical Formula 1,
R¹¹, R¹², R¹³, R¹⁴, R²¹, and R²² are each independently a methyl group or an ethyl group,
R³¹, R³², R³³ and R³⁴ are each independently a hydrogen atom, a cyano group, a nitro group, a fluoro group, a chloro group, a bromo group, an iodine group, a C₂F₅ group, an OCF₃ group, an SF₅ group, a C(O)CH₃ group, a C(O)OCH₃ group, a C(O)NH₂ group, or an SO₂CH₃ group,
A is F, Cl, Br or I, and
X¹¹, X¹², X¹³, X¹⁴, X¹⁵, X¹⁶, X¹⁷, and X¹⁸ are each independently a hydrogen atom, a cyano group, a C₂F₅ group, an OCF₃ group, an SF₅ group, a C(O)CH₃ group, a C(O)OCH₃ group, a C(O)NH₂ group, or an SO₂CH₃ group, but at least one selected from the group consisting of X¹¹, X¹², X¹³, X¹⁴, X¹⁵, X¹⁶, X⁷, and X¹⁸ is not a hydrogen atom.

According to the present disclosure, it is possible to provide a means of enabling the formation of a plating film with excellent filling properties and high flatness of the concave portion of the plating object.

### BRIEF DESCRIPTION OF THE DRAWINGS

FIG. 1 is a schematic diagram explaining the recess amount of via conductor according to an embodiment of the present disclosure.

### DETAILED DESCRIPTION OF EMBODIMENTS

Hereinafter, non-limiting embodiments of the present disclosure will be described. However, the present disclosure is not limited to the described embodiments and may be variously modified within the scope of the patent claims. In addition, embodiments described in this specification may be formed into other embodiments by arbitrarily combining them. In this specification, X-Y indicating a range is used to mean including the numerical values (X and Y) as lower and upper limits, and means “greater than or equal to X and less than or equal to Y”. In the present specification, unless otherwise specified, the operation, physical properties, etc. are measured under conditions of room temperature (20-25° C.)/relative humidity of 40 to 50% RH. In the present specification, A or B means including each of A and B. In the present specification, A and B means including combination of A and B.

The present inventive concept provides an ammonium salt compound that may be used as an additive for electrolytic copper plating or in an electrolytic copper plating solution composition which enables the formation of a plating film with excellent filling properties and high flatness, such as on the concave portion of a plating object.

### <Ammonium Salt Compound>

One embodiment of the present disclosure relates to an ammonium salt compound represented by the following Chemical Formula 1.

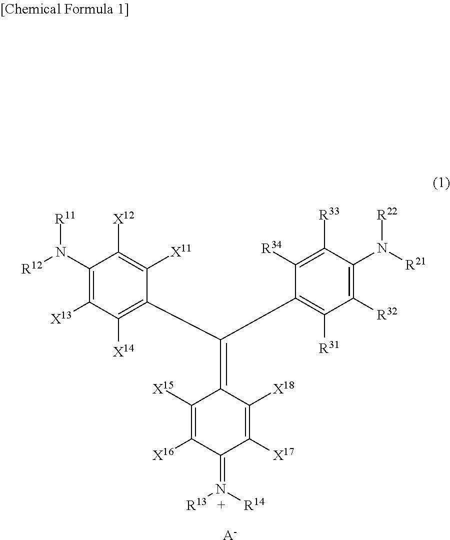

In an embodiment, in the chemical formula (1),
R¹¹, R¹², R¹³, R¹⁴, R²¹ and R²² are each independently a methyl group or an ethyl group,
R³¹, R³², R³³ and R³⁴ are each independently a hydrogen atom, a cyano group (CN group), a nitro group (NO₂ group), a fluoro group, a chloro group, a bromo group, an iodine group, a C₂F₅ group, an OCF₃ group, an SF₅ group, a C(O)CH₃ group, a C(O)OCH₃ group, a C(O)NH₂ group or an SO₂CH₃ group,
A is F, Cl, Br or I, and
X¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)CH₃ group, a C(O)OCH₃ group, a C(O)NH₂ group, or an SO₂CH₃ group, but at least one selected from the group consisting of X¹¹, X¹², X¹³, X¹⁴, X¹⁵, X¹⁶, X¹⁷ and X¹⁸ is not a hydrogen atom.

The OCF₃ group is a trifluoromethoxy group.

The C(O)CH₃ group is also expressed as a C(═O)—CH₃ group, the C(O)OCH₃ group is also expressed as a C(═O)—O—CH₃ group, the C(O)NH₂ group is also expressed as a C(═O)—NH₂ group, and the SO₂CH₃ group is also expressed as a S(═O)₂—CH₃ group.

In this specification, the object to be plated is referred to as the “plating object”, and the plating object that has been plated due to a plating process is referred to as the “plated object”.

The ammonium salt compound represented by the Chemical Formula 1 is preferably used in plating (e.g., a plating process) and is more preferably used in copper plating (e.g., a copper plating process). In this specification, the term copper plating refers to a general term for a plating process that deposits copper on a plating object and for plating that deposits a copper alloy on a plating object. The ammonium salt compound represented by the Chemical Formula 1 may be used for plating to form a copper film or for plating to form a copper alloy film. Elements other than copper contained in the copper alloy film are not necessarily limited. Copper alloys may contain elements other than copper, such as metals like aluminum, beryllium, zinc, nickel, tin, lead, and phosphorus.

In the Chemical Formula 1, X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group, a C(O)CH₃ group or an SO₂CH₃ group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group, or an SO₂CH₃ group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group, or an SO₂CH₃ group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an SF₅ group, or an SO₂CH₃ group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, or an SF₅ group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, an SF₅ group, or a cyano group, and, in an embodiment, at least one selected from the group consisting of X¹¹, X¹², X¹³, X¹⁴, X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom or a SF₅ group, and, in an embodiment, at least one selected from the group consisting of X¹¹, X¹², X¹³, X¹⁴, X¹⁵, X¹⁶, X¹⁷ and X¹⁸ is not a hydrogen atom.

In an embodiment, in the Chemical Formula 1, at least two selected from the group consisting of X¹¹, X¹², X¹³, X¹⁴, X¹⁵, X¹⁶, X¹⁷ and X¹⁸ are not hydrogen atoms. In an embodiment, at least six selected from the group consisting of X¹¹, X¹², X¹³, X¹⁴, X¹⁵, X¹⁶, X¹⁷ and X¹⁸ are not hydrogen atoms. In an embodiment, each of X¹¹, X¹², X¹³, X¹⁴, X¹⁵, X¹⁶, X¹⁷ and X¹⁸ are not hydrogen atoms.

In an embodiment, in the Chemical Formula 1, at least one selected from the group consisting of X¹¹, X¹², X¹³, and X¹⁴ is not a hydrogen atom. In an embodiment, at least one selected from the group consisting of X¹⁵, X¹⁶, X¹⁷ and X¹⁸ is not a hydrogen atom. In an embodiment, at least three selected from the group consisting of X¹¹, X¹², X¹³, and X¹⁴ are not hydrogen atoms. In an embodiment, at least three selected from the group consisting of X¹⁵, X¹⁶, X¹⁷ and X¹⁸ are not hydrogen atoms. In an embodiment, each of X¹¹, X¹², X¹³, X¹⁴, X¹⁵, X¹⁶, X¹⁷ and X¹⁸ are not hydrogen atoms.

In an embodiment, in the Chemical Formula 1, X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group, a C(O)CH₃ group or an SO₂CH₃ group, and at least one selected from the group consisting of X¹¹, X¹², X¹³, and X¹⁴ is not a hydrogen atom, and at least one selected from the group consisting of X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group or an SO₂CH₃ group. In an embodiment, at least one selected from the group consisting of X¹¹, X¹², X¹³, and X¹⁴ is not a hydrogen atom, and at least one selected from the group consisting X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group or an SO₂CH₃ group. In an embodiment, at least one selected from the group consisting of X¹¹, X¹², X¹³, and X¹⁴ is not a hydrogen atom, and at least one selected from the group consisting of X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group, an SF₅ group or an SO₂CH₃ group. In an embodiment, at least one selected from the group consisting of X¹¹, X¹², X¹³, and X¹⁴ is not a hydrogen atom, and at least one selected from the group consisting of X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group or an SF₅ group. In an embodiment, at least one selected from the group consisting of X¹¹, X¹², X¹³, and X¹⁴ is not a hydrogen atom, and at least one selected from the group consisting of X¹⁵, X¹⁶, X¹⁷ and X¹⁸ is not a hydrogen atom. X¹¹, X¹², X¹³, X¹⁴, X¹⁵, X¹⁶, X¹⁷ and X¹⁸ are each independently a hydrogen atom, a cyano group, a C₂F₅ group or a SF₅ group. In an embodiment, at least three selected from the group consisting of X¹¹, X¹², X¹³, and X¹⁴ are not hydrogen atoms, and at least three selected from the group consisting of X¹⁵, X¹⁶, X¹⁷ and X¹⁸ are not hydrogen atoms. X¹¹, X¹², X¹³, X¹⁴, X¹⁵, X¹⁶, X¹⁷ and X¹⁸ are each independently a hydrogen atom, an SF₅ group or a cyano group. In an embodiment at least three selected from the group consisting of X¹¹, X¹², X¹³, and X¹⁴ are not hydrogen atoms, and at least three selected from the group consisting of X¹⁵, X¹⁶, X¹⁷ and X¹⁸ are not hydrogen atoms. In an embodiment, X¹¹, X¹², X¹³, X¹⁴, X¹⁵, X¹⁶, X¹⁷ and X¹⁸ are each independently an SF₅ group or a cyano group.

In an embodiment, X¹¹, X¹², X¹³, X¹⁴, X¹⁵, X¹⁶, X¹⁷ and X¹⁸ are SF₅ groups.

In an embodiment, in the Chemical Formula 1, R¹¹, R¹², R¹³ and R¹⁴ are each independently a methyl group. R¹¹, R¹², R¹³ and R¹⁴ may be the same or different. In an embodiment, R¹¹, R¹², R¹³ and R¹⁴ are methyl groups, or R¹¹, R¹², R¹³ and R¹⁴ are ethyl groups.

In an embodiment, in Chemical Formula 1, R²¹ and R²², each independently, are preferably methyl groups. R²¹ and R²² may be the same or different. In an embodiment, R²¹ and R²² are methyl groups, or R²¹ and R²² are ethyl groups.

In an embodiment, in the Chemical Formula 1, R³¹, R³², R³³ and R³⁴ are each independently a hydrogen atom, a cyano group, a C₂F₅ group, an OCF₃ group, an SF₅ group, a C(O)CH₃ group, a C(O)OCH₃ group, a C(O)NH₂ group or an SO₂CH₃ group. R³¹, R³², R³³ and R³⁴ may be the same or different. For example, R³¹, R³², R³³ and R³⁴ may each independent be a hydrogen atom, a cyano group, a nitro group, a fluoro group, a chloro group, a bromo group, an iodine group, a C₂F₅ group, an OCF₃ group, an SF₅ group, a C(O)CH₃ group, a C(O)OCH₃ group, a C(O)NH₂ group or an SO₂CH₃ group.

In an embodiment, in Chemical Formula 1, A is fluorine F, chlorine Cl, bromine Br, or iodine I. For example, in an embodiment A is Cl, Br or I. In an embodiment, A is Cl or Br. In an embodiment, A is Cl. Therefore, in Chemical Formula 1, A- is a fluoride ion F—, a chloride ion Cl—, a bromide ion Br—, or an iodide ion I—. In an embodiment, A- is Cl—, Br— or I—. In an embodiment, A- is Cl— or Br—. In an embodiment, A- is Cl—.

In an embodiment, an ammonium salt compound according to an embodiment is an example of compound represented by the Chemical Formula 1, wherein R¹¹, R¹², R¹³, R¹⁴, R²¹ and R²² are each independently a methyl group or an ethyl group, R³¹, R³², R³³ and R³⁴ are a hydrogen atom, A is Cl, Br or I, X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)CH₃ group, a C(O)NH₂ group or an SO₂CH₃ group, and at least one selected from the group consisting of X¹¹, X¹², X¹³ and X¹⁴ is not a hydrogen atom, and at least one selected from the group consisting of X¹⁵, X¹⁶, X¹⁷ and X¹⁸ is not a hydrogen atom. An ammonium salt compound according to an embodiment is an example of a compound represented by the formula 1, wherein R¹¹, R¹², R¹³, R¹⁴, R²¹ and R²² are each independently a methyl group or an ethyl group, R³¹, R³², R³³ and R³⁴ are hydrogen atoms, A is Cl or Br, X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group or an SO₂CH₃ group, and at least one selected from the group consisting of X¹¹, X¹², X¹³ and X¹⁴ is not a hydrogen atom, and at least one selected from the group consisting of X¹⁵, X¹⁶, X¹⁷ and X¹⁸ is not a hydrogen atom. In an ammonium salt compound according to an embodiment, there may be a compound represented by the Chemical Formula 1, wherein R¹¹, R¹², R¹³ and R¹⁴ are each independently a methyl group or an ethyl group, R²¹ and R²² are each a methyl group R³¹, R³², R³³ and R³⁴ are each a hydrogen atom, A is Cl or Br, X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)NH₂ group or an SO₂CH₃ group, and at least one selected from the group consisting of X¹¹, X¹², X¹³ and X¹⁴ is not a hydrogen atom, and at least one selected from the group consisting of X¹⁵, X¹⁶, X¹⁷ and X¹⁸ is not a hydrogen atom. In an ammonium salt compound according to an embodiment, there may be a compound represented by the Chemical Formula 1, wherein R¹¹, R¹², R¹³ and R¹⁴ are each independently a methyl group or an ethyl group, R²¹ and R²² are methyl groups, R³¹, R³², R³³ and R³⁴ are hydrogen atoms, A is Cl or Br, X¹¹, X¹², X¹³, X¹⁴, X¹⁵, X¹⁶, X¹⁷ and X¹⁸ are each independently a hydrogen atom, a cyano group, a C₂F₅ group, a SF₅ group or an SO₂CH₃ group, and at least one selected from the group consisting of X¹¹, X¹², X¹³ and X¹⁴ is not a hydrogen atom, and at least one selected from the group consisting of X¹⁵, X¹⁶, X¹⁷ and X¹⁸ is not a hydrogen atom. In an ammonium salt compound according to an embodiment, there may be a compound represented by the Chemical Formula 1, wherein R¹¹, R¹², R¹³ and R¹⁴ are each independently a methyl group or an ethyl group, R²¹ and R²² are each a methyl group, R³¹, R³², R³³ and R³⁴ are a hydrogen atom, A is Cl, X¹¹, X¹², X¹³, X¹⁴, X¹⁵, X¹⁶, X¹⁷ and X¹⁸ are each independently a hydrogen atom, a cyano group, a C₂F₅ group or a SF₅ group, and at least three selected from the group consisting of X¹¹, X¹², X¹³ and X¹⁴ are not hydrogen atoms, and at least three selected from the group consisting of X¹⁵, X¹⁶, X¹⁷ and X¹⁸ are not hydrogen atoms. In an ammonium salt compound according to an embodiment, there may be, for example, a compound represented by the Chemical Formula 1, wherein R¹¹, R¹², R¹³, R¹⁴, R²¹ and R²² are methyl groups, R³¹, R³², R³³ and R³⁴ are hydrogen atoms, A is Cl, and X¹¹, X¹², X¹³, X¹⁴, X¹⁵, X¹⁶, X¹⁷ and X¹⁸ are each independently an SF₅ group or a cyano group.

In an embodiment, the following compound 1 to compound 9 may be specific examples of a compound of ammonium salt represented by Chemical Formula 1. The ammonium salt compound represented by Chemical Formula 1 may be any one of the following compound 1 to compound 9. For example, in some embodiments, the compound of ammonium salt represented by Chemical Formula 1 may be compounds 1, 2, 3, 4, 5, 7, 8 or 9, compounds 1, 2, 3, 4, 5, 8 or 9, compounds 1, 2, 3, 4, 5 or 9, compounds 1, 2, 3, 4 or 5, compounds 2, 4 or 5, compounds 2 or 5, or compound 5.

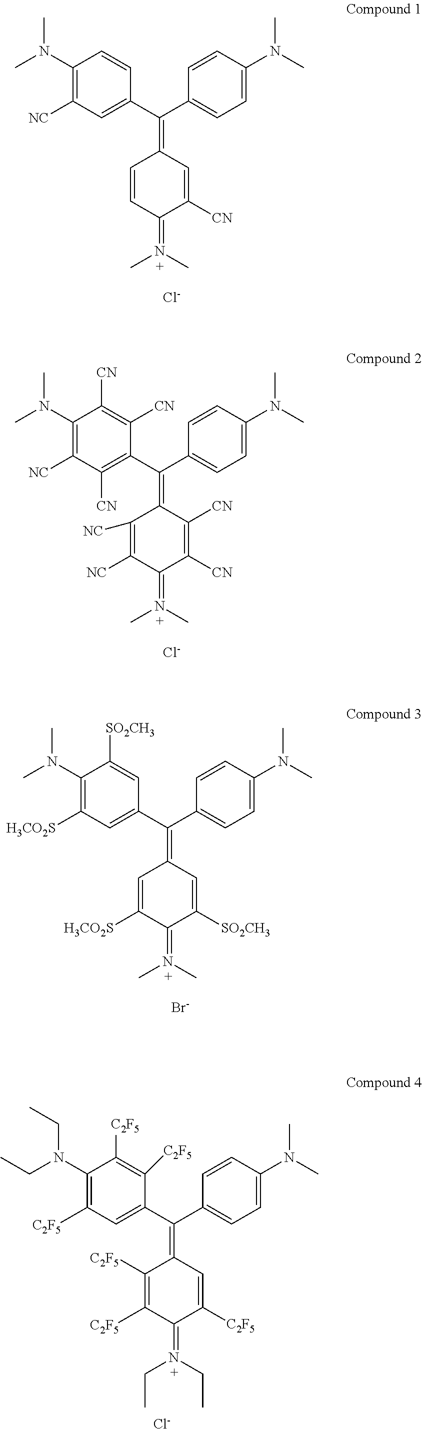

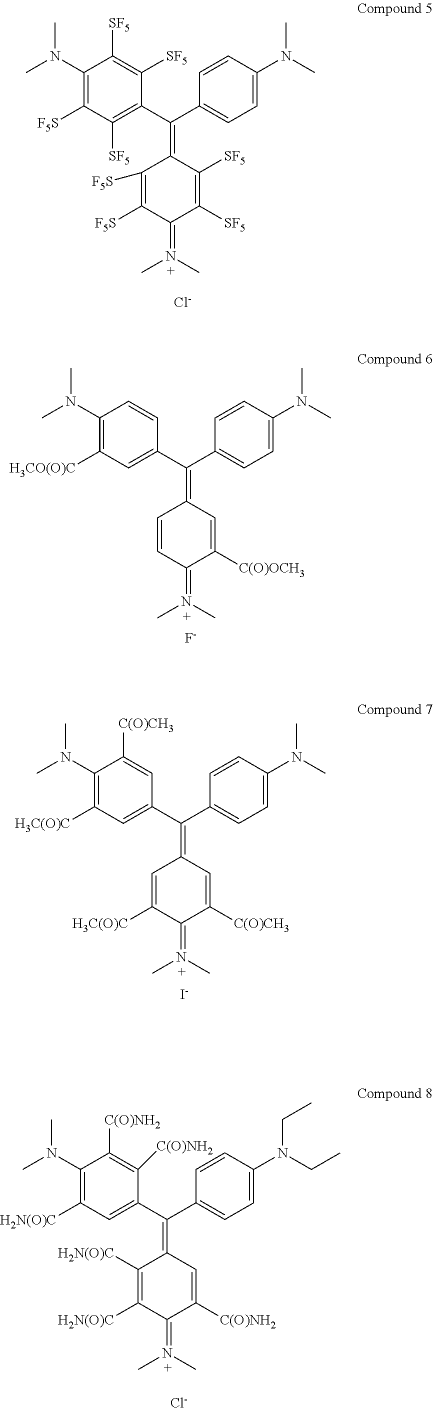

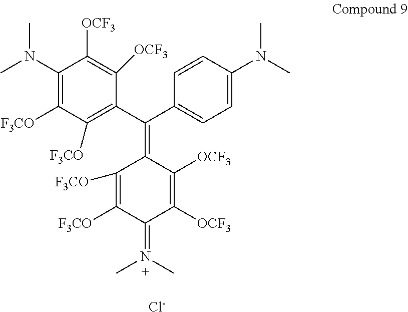

The ammonium salt compound represented by the Chemical Formula 1 may be synthesized based on the knowledge of known synthetic methods, but the synthetic method is not necessarily limited. In an embodiment, an ammonium salt compound according to one embodiment may be synthesized, for example, by changing raw materials or reaction conditions, etc., adding or excluding some of the sequences, and/or appropriately combining known synthetic methods, according to the method described in the examples. For example, compound 1 may be synthesized by the following reaction scheme 1. In the following reaction, p-toluenesulfonic acid and HCl are Brensted acid, and PbO₂ is an oxidizing agent.

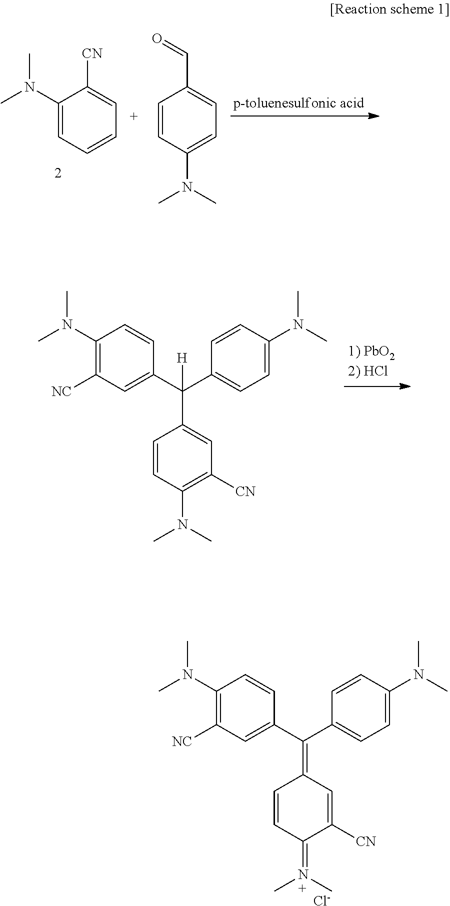

The method for confirming the structure of the ammonium salt compound represented by the Chemical Formula 1 is not necessarily limited, and a known method may be used. The structure of the ammonium salt compound represented by the above Chemical Formula 1 may be confirmed, for example, by ¹H-NMR, etc.

### <Additives for Electrolytic Copper Plating>

Another aspect of an embodiment of the present disclosure relates to an additive for electrolytic copper plating comprising an ammonium salt compound according to Chemical Formula 1. The additive for electrolytic copper plating according to an embodiment may be an additive for a pretreatment solution for electrolytic copper plating and/or an additive for an electrolytic copper plating solution composition. The ammonium salt compound according to Chemical Formula 1 may be a leveler for electrolytic copper plating. In this specification, a leveler refers to an additive that may increase the flatness of a plating film. In this specification, an electrolytic copper plating solution containing two or more substances is also referred to as an “electrolytic copper plating solution composition”. In some embodiments, an electrolytic copper plating additive comprises a compound selected from the group consisting of compound 1 to compound 9, a compound selected from the group consisting of compounds 1, 2, 3, 4, 5, 7, 8 and 9, a compound selected from the group consisting of compounds 1, 2, 3, 4, 5, 8 and 9, a compound selected from the group consisting of compounds 1, 2, 3, 4, 5 and 9, a compound selected from the group consisting of compounds 1, 2, 3, 4 and 5, a compound selected from the group consisting of compounds 2, 4 and 5, a compound selected from the group consisting of compounds 2 and 5, or compound 5.

The additive for electrolytic copper plating according to an embodiment may be an additive for electrolytic copper plating that forms a copper film, or an additive for electrolytic copper plating that forms a copper alloy film. Elements other than copper contained in the copper alloy film are not necessarily limited. Elements other than copper included in the copper alloy film may include, for example, elements such as those exemplified in the description of the copper alloy film described above.

### <Electrolytic Copper Plating Solution Composition>

Another aspect of an embodiment of the present disclosure relates to an electrolytic copper plating solution composition comprising a copper ion source and an ammonium salt compound according to Chemical Formula 1. The composition of the electrolytic copper plating solution according to an embodiment may contain a copper ion source, an ammonium salt compound according to Chemical Formula 1, and at least one component selected from the group consisting of an accelerator and an inhibitor.

The composition of the electrolytic copper plating solution according to an embodiment includes a copper ion source, an ammonium salt compound according to Chemical Formula 1, at least one component selected from the group consisting of an accelerator and an inhibitor, at least one component that is washed from a group consisting of a halide ion source and an acid other than the ammonium salt compound represented by the Chemical Formula 1, and water

In an embodiment, the electrolytic copper plating solution composition contains an accelerator and an inhibitor. In an embodiment the electrolytic copper plating solution composition contains a halide ion source, acid, and water. An electrolytic copper plating solution composition according to an embodiment of the present disclosure includes a copper ion source, an ammonium salt compound according to Chemical Formula 1, an accelerator, an inhibitor, a halide ion source, acid, and water.

The electrolytic copper plating solution composition according to an embodiment may be an electrolytic copper plating solution composition for forming a copper film or an electrolytic copper plating solution composition for forming a copper alloy film. Elements other than copper contained in the copper alloy film are not necessarily limited. Elements other than copper included in the copper alloy film include, for example, the elements described for the copper alloy film.

### (Ammonium Salt Compound)

An electrolytic copper plating solution composition according to an embodiment of the present disclosure includes an ammonium salt compound represented by the Chemical Formula 1. In an embodiment, the compound represented by the Chemical Formula 1 may be used alone or in combination of two or more. The electrolytic copper plating solution composition according to an embodiment may contain at least one compound selected from the group consisting of compound 1 to compound 9 described above. The ammonium salt compound according to the aspect may be a leveler for electrolytic copper plating.

In some embodiments, the ammonium salt compound according to the above aspect included in the electrolytic copper plating solution composition is at least one compound selected from the group consisting of compound 1 to compound 9, at least one compound selected from the group consisting of compounds 1, 2, 3, 4, 5, 7, 8 and 9, at least one compound selected from the group consisting of compounds 1, 2, 3, 4, 5, 8 and 9, at least one compound selected from the group consisting of compounds 1, 2, 3, 4, 5 and 9, at least one compound selected from the group consisting of compounds 1, 2, 3, 4, 5, at least one compound selected from the group consisting of compounds 2, 4 and 5, at least one compound selected from the group consisting of compounds 2 or compound 5. For example, in an embodiment, the ammonium salt compound included in the electrolytic copper plating solution composition is at least one compound selected from the group consisting of compound 2 and compound 5 or compound 5.

The concentration of the ammonium salt compound represented by the Chemical Formula 1 in the electrolytic copper plating solution composition (mass of the ammonium salt compound represented by the Chemical Formula 1 per 1 L of the electrolytic copper plating solution composition) is not necessarily limited to, but may be in a range of 0.1 mg/L to 1000 mg/L, 1 mg/L to 100 mg/L, 5 mg/L to 50 mg/L, and 10 mg/L to 20 mg/L. The concentration of the ammonium salt compound represented by the Chemical Formula 1 in the electrolytic copper plating solution composition represents the total amount when the electrolytic copper plating solution composition contains two or more types of ammonium salt compounds represented by the Chemical Formula 1.

### (Copper Ion Source)

In an embodiment, as a copper ion source, although not necessarily limited thereto, may be, for example, copper salt. In an embodiment, the copper salt may be a copper salt that ionizes to generate copper (II) ions. The copper salt is not necessarily limited to, but may include, for example, copper sulfate, copper halides, copper acetate, copper nitrate, copper tetrafluoroborate, copper alkylsulfonate, copper arylsulfonate, copper sulfamate, copper perchlorate, copper gluconate, and copper citrate. Copper halide is not necessarily limited to, but includes, for example, copper chloride. The copper alkylsulfonates are not necessarily limited to, but may include, for example, copper methanesulfonate, copper ethanesulfonate, and copper propanesulfonate. The copper arylsulfonates are not necessarily limited to, but may include, for example, copper benzenesulfonate, copper p-toluenesulfonate, etc. The copper ion source may be one type alone, or two or more types may be used together. The copper ion source may include at least one selected from the group consisting of the compounds exemplified above. In an embodiment, the copper ion source is at least one selected from the group consisting of compounds exemplified above, such as a copper salt that generates copper (II) ions by ionization, for example, copper sulfate.

The concentration of the copper ion source in the electrolytic copper plating solution composition (mass of the copper ion source per 1 L of the electrolytic copper plating solution composition) is not necessarily limited to, but may be in a range of 1 g/L to 500 g/L, 10 g/L to 400 g/L, 50 g/L to 300 g/L, and 100 g/L to 200 g/L. The concentration of the copper ion source in the electrolytic copper plating solution composition represents the total amount when the electrolytic copper plating solution composition includes two or more copper ion sources.

### (Accelerator)

The electrolytic copper plating solution composition according to an embodiment of the present disclosure may additionally contain an accelerator or may not contain an accelerator. In this specification, an accelerator refers to an additive that may increase the plating speed of electrolytic copper plating. The accelerator is a compound different from the ammonium salt compound represented by the Chemical Formula 1. The accelerator is not necessarily limited to, but includes, for example, a thiol compound, a disulfide compound, and the like. In an embodiment, as a specific example, although not necessarily limited thereto, bis(3-sulfopropyl)disulfide, 3-(benzothiazolyl-2-thio) propylsulfonic acid, 3-mercaptopropane-1-sulfonic acid, N,N-dimethyldithiocarbaminic acid (3-sulfopropyl) ester, ethylenedithiodipropylsulfonic acid, bis(p-sulfophenyl)disulfide, a salt thereof, etc. may be exemplified. The accelerator may be one type alone, or two or more types may be used in combination. The accelerator may include at least one selected from the group consisting of the compounds exemplified above. In an embodiment, the accelerator is at least one selected from the group consisting of compounds exemplified above, a disulfide compound, or bis(3-sulfopropyl)disulfide (e.g., bis(3-sulfopropyl)persulfide, abbreviation: SPS).

The concentration of the accelerator in the electrolytic copper plating solution composition (e.g., mass of the accelerator per 1 L of the electrolytic copper plating solution composition) is not necessarily limited. In an embodiment, the concentration of the accelerator is in a range of 0.1 mg/L to 1000 mg/L, 0.5 mg/L to 100 mg/L, 1 mg/L to 50 mg/L, or 5 mg/L to 15 mg/L. The concentration of the accelerator in the electrolytic copper plating solution composition represents the total amount when the electrolytic copper plating solution composition includes two or more accelerators.

### (Inhibitor)

The electrolytic copper plating solution composition according to an embodiment may further include an inhibitor, or may not include an inhibitor. In this specification, an inhibitor refers to an additive capable of suppressing the plating speed of electrolytic copper plating. The inhibitor is a compound different from the ammonium salt compound represented by the Chemical Formula 1. In an embodiment, the inhibitor is not necessarily limited thereto, but includes, for example, polyalkylene glycol, stearic acid polyglycol ester, oleic acid polyglycol ester, stearyl alcohol polyglycol ether, nonylphenol polyglycol ether, octanol polyalkylene glycol ether, etc. Examples of the polyalkylene glycol include, but are not necessarily limited to, polyethylene glycol, polypropylene glycol, ethylene glycol-propylene glycol copolymer, and the like. The inhibitor may be one type alone, or two or more types may be used together. The inhibitor may include at least one selected from the group consisting of the compounds exemplified above. In an embodiment the inhibitor is at least one selected from the group consisting of the compounds exemplified above, such as a polyalkylene glycol. For example, in an embodiment, the inhibitor may be at least one selected from the group consisting of polyethylene glycol, polypropylene glycol and ethylene glycol-propylene glycol copolymer. In an embodiment, the inhibitor may be a polyethylene glycol.

The concentration of the inhibitor in the electrolytic copper plating solution composition (e.g., mass of the inhibitor per 1 L of the electrolytic copper plating solution composition) is not necessarily limited to, but may be in a range of 0.1 mg/L to 1,000 mg/L, 100 mg/L to 800 mg/L, 200 mg/L to 600 mg/L, and 300 mg/L to 400 mg/L. The concentration of the inhibitor in the electrolytic copper plating solution composition represents the total amount when the electrolytic copper plating solution composition contains two or more types of inhibitors.

### (Halide Ion Source)

The electrolytic copper plating solution composition according to an embodiment may further contain a halide ion source other than the ammonium salt compound represented by the Chemical Formula 1, or may not contain a halide ion source other than the ammonium salt compound represented by the Chemical Formula 1. In this specification, a halide ion source other than the ammonium salt compound represented by the Chemical Formula 1 is also simply referred to as “a halide ion source”. Examples of halide ion sources include, but are not necessarily limited to, fluoride ion (F⁻) sources, chloride ion (Cl⁻) sources, bromide ion (Br⁻) sources, and iodide ion (I⁻) sources. The halide ion source may be used alone or in combination of two or more types. The halide ion source may include at least one selected from the group consisting of a fluoride ion source, a chloride ion source, a bromide ion source, and an iodide ion source. Examples of the halide ion source are not necessarily limited to, but more specifically, hydrogen halide, alkali halide, and the like may be exemplified. Examples of hydrogen halides include, but are not necessarily limited to, hydrochloric acid (hydrogen chloride), hydrogen bromide, etc. Hydrogen halides may be used alone or in combination of two or more types.

Alkali halides are not necessarily limited to, but include, for example, chlorides of alkali metals such as sodium chloride and potassium chloride; bromides of alkali metals such as sodium bromide and potassium bromide. Alkali halides may be used alone or in combination of two or more types. The halide ion source may include at least one selected from the group consisting of compounds exemplified above. In an embodiment, the halide ion source may be at least one selected from the group consisting of a fluoride ion source, a chloride ion source, a bromide ion source, and an iodide ion source. In an embodiment, the halide ion source may be a chloride ion source. In an embodiment, the halide ion source may be at least one selected from the group consisting of hydrochloric acid (hydrogen chloride) and a chloride of an alkali metal. In an embodiment, the halide ion source may be a chloride of an alkali metal. In an embodiment, the halide ion source may be at least one selected from the group consisting of sodium chloride and potassium chloride. In an embodiment, the halide ion source may be sodium chloride.

The concentration of a halide ion source other than the ammonium salt compound represented by the Chemical Formula 1 in the electrolytic copper plating solution composition (e.g., the mass of the halide ion source other than the ammonium salt compound represented by the Chemical Formula 1 per 1 L of the electrolytic copper plating solution composition) is not necessarily limited to, but may be in a range of 0.1 mg/L to 1,000 mg/L, 1 mg/L to 500 mg/L, 10 mg/L to 250 mg/L, and 50 mg/L to 100 mg/L. The concentration of a halide ion source other than the ammonium salt compound represented by the Chemical Formula 1 in the electrolytic copper plating solution composition represents the total amount, when the electrolytic copper plating solution composition contains two or more types of halide ion sources other than the ammonium salt compound represented by the Chemical Formula 1.

### (Acid)

The electrolytic copper plating solution composition according to an embodiment may or may not include an acid. Examples of acids include, but are not necessarily limited to, inorganic acids, alkanesulfonic acids, substituted or unsubstituted alkanesulfonic acids, and substituted or unsubstituted arylsulfonic acids. In an embodiment, the inorganic acid is not necessarily limited to, but includes, for example, acetic acid, nitric acid, sulfuric acid, hydrochloric acid, borate hydrofluoric acid, hydrobromic acid, perchloric acid, chromic acid, phosphoric acid, and sulfamic acid. The substituted or unsubstituted alkanesulfonic acid is not necessarily limited to, but includes, for example, methanesulfonic acid, ethanesulfonic acid, propanesulfonic acid, and trifluromethanesulfonic acid. The substituted or unsubstituted arylsulfonic acid is not necessarily limited to, but includes, for example, benzene sulfonic acid and p-toluenesulfonic acid. Acid may be alone, or two or more types may be used together. The acid may include at least one selected from the group consisting of the compounds exemplified above. In an embodiment, the acid is an inorganic acid, such as sulfuric acid.

The concentration of acid in the electrolytic copper plating solution composition (e.g., the mass of acid per 1 L of the electrolytic copper plating solution composition) is not necessarily limited to, but may be in a range of 1 g/L to 500 g/L, 10 g/L to 400 g/L, 50 g/L to 300 g/L, and 100 g/L to 200 g/L. The concentration of acid in the electrolytic copper plating solution composition represents the total amount when the electrolytic copper plating solution composition contains two or more types of acid.

### (Water)

The electrolytic copper plating solution composition in accordance with an embodiment may or may not contain water additionally.

(Metal Ion Source Other than Copper Ion Source)

The electrolytic copper plating solution composition according to an embodiment may further include a metal ion source other than a copper ion source or does not have to include a metal ion source other than a copper ion source. In the present specification, metal ion sources other than copper ion sources indicate materials that are metal ion sources other than copper ion sources and do not correspond to the ammonium salt compound represented by the Chemical Formula 1, the copper ion source, the halide ion source, the accelerator, the inhibitor, and the acid. In an embodiment, metal ion sources other than copper may be used in combination with a copper ion source when a copper alloy film is formed. As a metal ion source other than the copper ion source, there are no particular limitations, but examples thereof include metal ion sources of metal elements as exemplified in the description of the copper alloy film described above. In an embodiment, as metal ion sources other than copper ion sources, for example, tin ion sources such as tin salts may be used. The tin salt is not necessarily limited to, but includes, for example, tin sulfate, tin alkanesulfonic acid, tin arylsulfonic acid, and the like. The tin ion source may be one type alone, or two or more types may be used in combination. Metal ion sources other than copper ion sources may be used alone, or two or more types may be used together. The concentration of metal ion sources other than copper ion sources in the electrolytic copper plating solution composition is not necessarily limited to, and the concentration at which a desired plating film is obtained may be used.

### (Other Component)

The electrolytic copper plating solution composition in accordance with an embodiment may further include components other than those exemplified above or may not further include other components.

### (Manufacturing Method of Electrolytic Copper Plating Solution Composition)

The method for producing an electrolytic copper plating solution composition according to an embodiment of the present disclosure is not necessarily limited. The method for producing an electrolytic copper plating solution composition according to an embodiment of the present disclosure may be produced by mixing each component in any order. In an embodiment, a manufacturing method includes, for example, a method including a step of first adding one or more inorganic substance to a plating bath, and then adding one or more organic substance to the plating bath. An example of a manufacturing method includes a method including a step of mixing a plurality of inorganic substances an inorganic copper ion source, an inorganic acid, a inorganic halide ion source other than the ammonium salt compound represented by the Chemical Formula 1 and water, and then adding a plurality of organic substances including the ammonium salt compound represented by the Chemical Formula 1, an organic accelerator, and an organic inhibitor to the obtained mixture. An example of a manufacturing method includes a method including the steps of mixing an inorganic copper ion source, an inorganic acid, an inorganic halide ion source other than the ammonium salt compound represented by the Chemical Formula 1, and water, and then adding an ammonium salt compound represented by the Chemical Formula 1, an organic accelerator, and an organic inhibitor to the obtained mixture. However, the mixing method and conditions, etc. are not necessarily limited thereto.

### <Electrolytic Copper Plating Method>

Another aspect of the present disclosure relates to an electrolytic copper plating method including the step of obtaining a plated product using the electrolytic copper plating solution composition according to the aspect. An electrolytic copper plating method according to an embodiment includes a step of forming a via conductor and a wiring pattern on a plated product using the electrolytic copper plating solution composition according to an embodiment of the present disclosure. In the electrolytic copper plating method according to an embodiment of the present disclosure, an electrolytic copper plating solution composition according to an embodiment may be used.

The electrolytic copper plating method according to an embodiment may be an electrolytic copper plating method for forming a copper film or an electrolytic copper plating method for forming a copper alloy film. Elements other than copper contained in the copper alloy film are not necessarily limited. In an embodiment, elements other than copper included in the copper alloy film may include, for example, the elements exemplified as examples in the description of the copper alloy film discussed above.

The electrolytic copper plating method is not necessarily limited to but may be a method including the steps of contacting a plating object with an electrolytic copper plating solution composition according to the above aspect, applying current, and electrolytically plating copper or a copper alloy on the plating object. The electrolytic copper plating method is not necessarily limited to but may be a method including the steps of contacting a plating object with an electrolytic copper plating solution composition according to the above aspect, applying current, and electrolytically plating copper on the plating object. The method of bringing the plating object into contact with the electrolytic copper plating solution composition is not necessarily limited to, and examples thereof include a method of immersing the plating object in the electrolytic copper plating solution composition.

In an embodiment, before plating, the plating object may be subjected to cleaning, rinsing and/or pretreatment. Pretreatment is not necessarily limited to, but may include, for example, acid activation treatment.

During plating, the plating object and at least one anode are connected to a current source or a voltage source. Additionally, during the plating process, the plating object and at least one anode are placed in contact with the electrolytic copper plating solution composition according to the above aspect. Typically, the plating object functions as a cathode. The anode may be a soluble anode such as a copper anode and/or an insoluble anode. When a current is applied, copper or a copper alloy is deposited on the plating object (e.g., at least on top of a part of the plating object). In an embodiment, in the plating object, the via and/or the trench may be filled to form a via conductor and a wiring pattern in the plated product. During the plating treatment, the electrolytic copper plating solution composition may be stirred. The stirring method is not necessarily limited, and a known stirring method may be used. In an embodiment, the stirring method may include, for example, air spraying, stirring of a workpiece, collision, and the like, but the stirring method is not necessarily limited to these methods. The cathode current density is not necessarily limited. In an embodiment, the anode current density may be in a range of 0.05 A/dm² to 10 A/dm², but the anode current density is not necessarily limited to a value within this range. The temperature of the electrolytic copper plating solution composition (e.g., the temperature of the plating bath) is not necessarily limited to, but may be a temperature within the range of 10° C. to 65° C., 10° C. to 35° C., and 15° C. to 30° C. The plating treatment time is not necessarily limited to, but may be, for example, a time in the range of 1 minute to 100 hours, 5 minutes to 10 hours, and the like.

As for the plating object, there are no special restrictions. In an embodiment, the plating object may be a substrate, such as a substrate having a rough structure such as a via and/or a trench. Specific examples of the uneven structure are not necessarily limited to, but include, for example, trenches, blind micro vias, through silicon vias, through holes, through glass vias, and the like. In an embodiment, the substrate may be a substrate selected from the group consisting of a printed circuit board, an IC substrate, a semiconductor wafer, a ceramic, and a glass substrate. The substrate may contain a conductor layer

The conductor layer is not necessarily limited to, but includes, for example, a copper film (e.g., a copper plating film) and a copper plate. The substrate may include a resin insulating film. In an embodiment, the resin insulating film may include, but is not necessarily limited to, polyimide films, etc. The substrate may include a metal seed layer. In an embodiment, the metal seed layer may include, but is not necessarily limited to, a copper seed layer, etc. The material of the substrate may include, but is not necessarily limited to, resin, ceramic, glass or silicon.

According to each aspect of the present disclosure described above, a means is provided that enables the formation of a plating film having excellent filling properties in a concave portion of a plating object and high flatness. According to each aspect of the present disclosure described above, there is provided a means for enabling the formation of a plating film with excellent filling properties and high flatness of the concave portion of plating object. According to an embodiment, provided is a means for enabling the formation of a plating film having excellent filling properties of vias and high flatness.

While non-limiting embodiments of the present disclosure have been described in detail, it is to be understood that they are illustrative and examples only and not restrictive, and that the scope of embodiments of the present disclosure are not limited thereto.

The present disclosure includes the following aspects and forms:
[1] An ammonium salt compound represented by the following chemical formula 1,

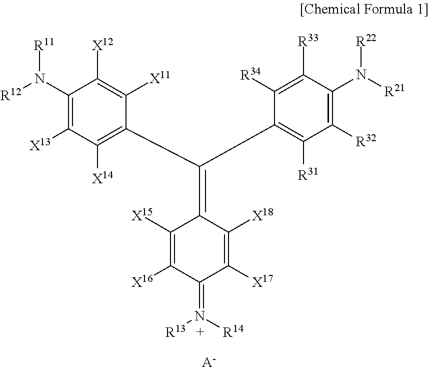

In Chemical Formula 1,
R¹¹, R¹², R¹³, R¹⁴, R²¹ and R²² are each independently a methyl group or an ethyl group,
R³¹, R³², R³³ and R³⁴ are each independently a hydrogen atom, a cyano group, a nitro group, a fluoro group, a chloro group, a bromo group, an iodine group, a C₂F₅ group, an OCF₃ group, an SF₅ group, a C(O)CH₃ group, a C(O)CH₃ group, a C(O)NH₂ group or an SO₂CH₃ group,
A is F, Cl, Br or I,
X¹¹, X¹², X¹³, X¹⁴, X¹⁵, X¹⁶, X¹⁷ and X¹⁸ are each independently a hydrogen atom, a cyano group, a C₂F₅ group, an OCF₃ group, an SF₅ group, a C(O)CH₃ group, a C(O)OCH₃ group, a C(O)NH₂ group or an SO₂CH₃ group, provided that at least one selected from the group consisting of X¹¹, X¹², X¹³, X¹⁴, X¹⁵, X¹⁶, X¹⁷ and X¹⁸ is not a hydrogen atom;
[2] X¹¹, X¹², X¹³, X¹⁴, X¹⁵, the X¹⁶, X¹⁷ and X¹⁸ are each independently a hydrogen atom, a cyano group, a C₂F₅ group, an OCF₃ group, an SF₅ group, a C(O)NH₂ group or an SO₂CH₃ group, and
   an ammonium salt compound as described in [1], wherein at least one selected from the group consisting of X¹¹, X¹², X¹³, X¹⁴, X¹⁵, X¹⁶, X¹⁷ and X¹⁸ is not a hydrogen atom;
[3] The A is an ammonium salt compound described in [1] or [2], which is Cl or Br;
[4] X¹¹, X¹², X¹³, X¹⁴, X¹⁵, X¹⁶, X¹⁷ and X¹⁸ are each independently a hydrogen atom, a cyano group, a C₂F₅ group, an SF₅ group, or an SO₂CH₃ group, and
   at least one selected from the group consisting of X¹¹, X¹², X¹³, X¹⁴, X⁵, X¹⁶, X¹⁷ and X¹⁸ is not a hydrogen atom, but an ammonium salt compound of any one of the claims [1] to [3];
[5]X¹¹, X¹², X¹³, X¹⁴, X¹⁵, X¹⁶, X¹⁷ and X¹⁸ are each independently a hydrogen atom, a cyano group, a C₂F₅ group, or an SF₅ group,
   At least three selected from the group consisting of X¹¹, X¹², X¹³ and X¹⁴, and at least three selected from the group consisting of X¹⁵, X¹⁶, X¹⁷ and X¹⁸ are not hydrogen atoms, but ammonium salt compounds of any one of the claims [1] to [4];
[6] An ammonium salt compound according to any one of [1] to [5], wherein R¹¹, R¹², R¹³ and R¹⁴ are methyl groups;
[7] The ammonium salt compound described in any one of [1] to [6], wherein R²¹ and R²² are methyl groups;
[8] The ammonium salt compounds described in [1] to [7], wherein R³¹, R³², R³³ and R³⁴ are hydrogen atoms;
[9] An ammonium salt compound described in [1], which is any one of compound 1 to compound 9;
[10] An additive for electrolytic copper plating comprising an ammonium salt compound as described in any one of [1] to [9];
[11] An electrolytic copper plating solution composition comprising: copper ion source and ammonium salt compound described in any one of the above [1] to [9]:
[12] An electrolytic copper plating solution composition as described in [11], further comprising at least one component selected from the group consisting of an accelerator and an inhibitor;
[13] An electrolytic copper plating method, comprising a step of forming a via conductor and a wiring pattern on a plated product using the electrolytic copper plating solution composition described in [11] or [12].

### Embodiment

The effects of the present disclosure are explained using the following examples and comparative examples. However, the technical scope of embodiments of the present disclosure is not limited to the following examples. Additionally, unless otherwise specified, “%” and “part” mean “mass %” and “mass part”, respectively.

### <Synthesis of Ammonium Salt Compounds>

### (Compound 1)

4-(Dimethylamino)benzaldehyde (compound R1-2 below) (10 mmol), 2-(dimethylamino)benzonitrile (compound R1-1 below) (40 mmol), p-toluenesulfonic acid (10 mmol), and benzene (20 mL) were placed in a flask equipped with a condenser and reacted at 100° C. for 9 hours. The reaction mixture was diluted with benzene (40 mL), washed with a concentration of 10 mass % sodium bicarbonate solution, and subsequently washed with saturated saline. After removing the solvent from the obtained mixture, the residue was purified by silica gel column chromatography (dissolving solution: mixture of hexane and ethyl acetate, hexane:ethyl acetate=10:1 (volume ratio)) to obtain compound P1. The yield of compound P1 in this reaction was 95% when rounded to nearest the first decimal place.

The obtained compound P1 (0.5 mmol) was dissolved in 3 mL of ion-exchanged water, PbO₂ (0.65 mmol) and 15 drops of concentrated HCl (12 N (concentration 37 mass %) HCl aqueous solution) were added dropwise, and the mixture was stirred at room temperature overnight. The obtained reaction solution was extracted using a mixture of methanol (30 mL), concentrated HCl (12 N (concentration 37 mass %) HCl aqueous solution) (3.78 mL), and methylene chloride (20 mL), and the obtained extract was concentrated. The obtained concentrate was purified by silica gel chromatography (dissolution solution: mixture of methylene chloride and methanol, methylene chloride:methanol=9:1 (volume ratio)) to obtain compound 1. The yield of compound 1 in this reaction was 100% when rounded to the nearest first decimal place.

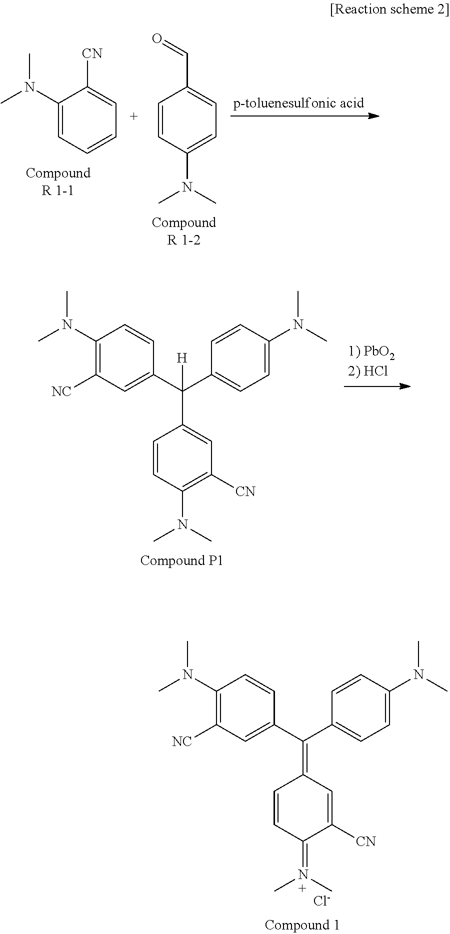

The structure of the obtained compound 1 was identified by nuclear magnetic resonance (¹H-NMR):

¹H-NMR (300 MHz, CDCl₃) σ=8.10 (s, 1H), 7.29 (s, 1H), 7.19-7.12 (m, 3H), 6.80-6.74 (m, 2H), 6.67-6.60 (m, 1H), 6.20-6.13 (m, 1H), 5.96-5.87 (m, 1H), 3.02 (s, 6H), 3.01 (s, 6H), 0.90 (s, 6H) ppm.

### (Compound 2)

Compound 2 was obtained by the same manufacturing method as the manufacturing method of compound 1 in embodiment 1, except that 2-(dimethylamino)benzonitrile (40 mmol) is changed to the same molar amount of (dimethylamino)benzene-1,2,4,5-tetracarbonitrile (compound R2-1 below).

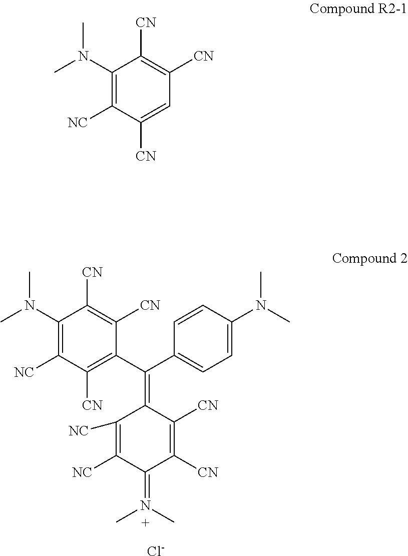

The structure of the obtained compound 2 was identified by nuclear magnetic resonance (¹H-NMR):

¹H-NMR (300 MHz, CDCl₃) σ=7.21-7.13 (m, 2H), 6.84-6.73 (m, 2H), 3.03 (s, 6H), 3.00 (s, 6H), 0.94 (s, 6H) ppm.

### (Compound 3)

2-(Dimethylamino)benzonitrile (40 mmol) was replaced with the same molar amount of N,N-dimethyl-2,6-bis(methylsulfonyl)aniline (compound R3-1 below), and PbO2 (0.65 mmol) and 15 drops of concentrated HCl (12 N (concentration 37 mass %) HCl aqueous solution) were added dropwise, and PbO2 (0.65 mmol) and 15 drops of HBr aqueous solution (concentration 47.0 to mass %) were added dropwise, thereby obtaining compound 3 by the same production method as that of compound 1 in Example 1.

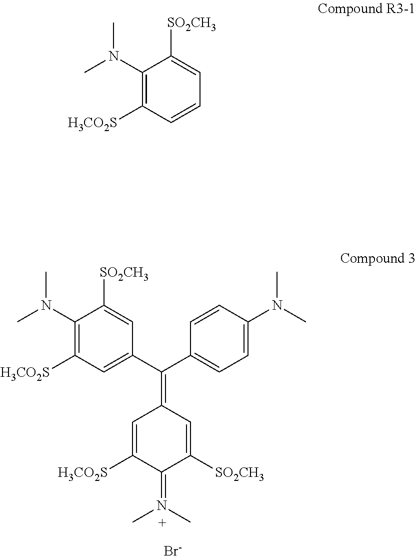

The structure of the obtained compound 3 was identified by nuclear magnetic resonance (¹H-NMR):

¹H-NMR (300 MHz, CDCl₃) σ=8.65 (s, 2H), 7.96 (s, 2H), 7.20-7.11 (m, 2H), 6.84-6.74 (m, 2H), 3.32 (s, 6H), 3.02 (s, 6H), 3.01 (s, 6H), 2.84 (s, 6H), 0.90 (s, 6H) ppm.

### (Compound 4)

Compound 4 was obtained by the same manufacturing method as the manufacturing method for compound 1 of Example 1, except that 2-(dimethylamino)benzonitrile (40 mmol) was replaced with the same molar amount of N,N-diethyl-2,3,6-tris(perfluoroethyl)aniline (compound R4-1 below).

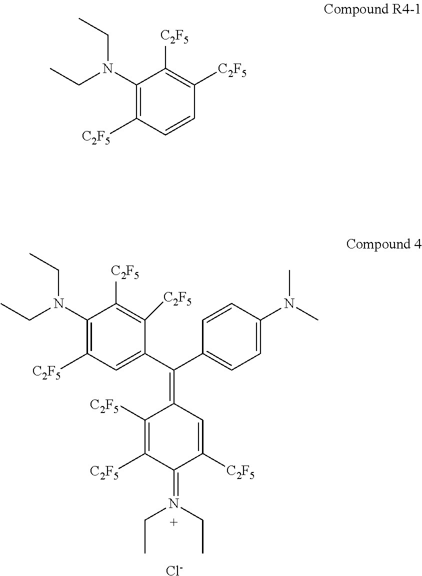

The structure of the obtained compound 4 was identified by nuclear magnetic resonance (1H-NMR):

¹H-NMR (300 MHz, CDCl₃) σ=7.81 (s, 1H), 7.21 (s, 1H), 7.19-7.12 (m, 2H), 6.80-6.70 (m, 2H), 4.07 (q, J=7.1 Hz, 4H), 3.40 (q, J=7.0 Hz, 4H), 1.41 (t, J=7.3 Hz, 6H), 1.12 (t, J=7.3 Hz, 6H), 0.93 (s, 6H) ppm.

### (Compound 5)

Compound 5 was obtained by the same manufacturing method as the manufacturing method for compound 1 of Example 1, except that 2-(dimethylamino)benzonitrile (40 mmol) was replaced with an equal molar amount of N,N-dimethyl-2,3,5,6-tetrakis(pentafluoro-λ6-sulfanyl)aniline (compound R5-1 below).

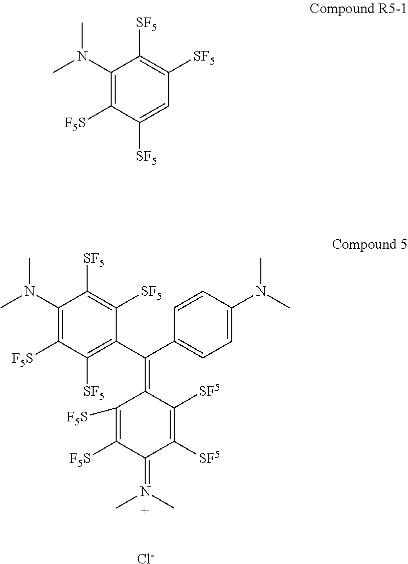

The structure of the obtained compound 5 was identified by nuclear magnetic resonance (¹H-NMR):

¹H-NMR (300 MHz, CDCl₃) σ=7.20-7.12 (m, 2H), 6.86-6.75 (m, 2H), 3.02 (s, 6H), 3.00 (s, 6H), 0.92 (s, 6H) ppm.

### (Compound 6)

2-(Dimethylamino)benzonitrile (40 mmol) was changed to methyl 2-(dimethylamino)benzoate (compound R6-1 below) in the same molar amount, and PbO₂ (0.65 mmol) and 15 drops of concentrated HCl (12 N (concentration 37 mass %) HCl aqueous solution) were added dropwise, and PbO₂ (0.65 mmol) and 15 drops of HF aqueous solution (concentration 46.0 to 48.0 mass %) were added dropwise, thereby obtaining compound 6 by the same production method as that of compound 1 in Example 1.

The structure of the obtained compound 6 was identified by nuclear magnetic resonance (¹H-NMR):

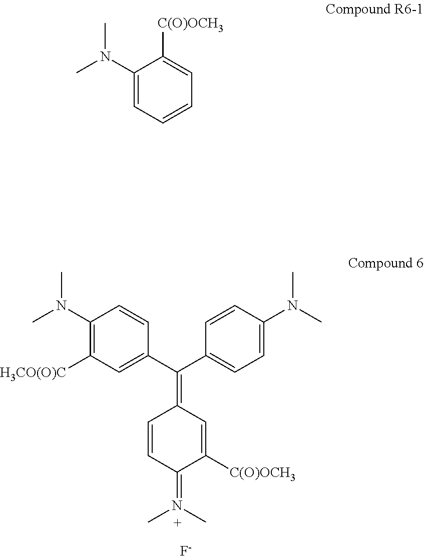

¹H-NMR (300 MHz, CDCl₃) σ=8.50 (s, 1H), 7.37 (s, 1H), 7.24-7.07 (m, 3H), 6.89-6.78 (m, 3H), 6.20-6.13 (m, 1H), 5.88-5.70 (m, 1H), 3.95 (s, 3H), 3.71 (s, 3H), 3.00 (s, 6H), 2.93 (s, 6H), 0.88 (s, 6H) ppm.

### (Compound 7)

2-(Dimethylamino)benzonitrile (40 mmol) was replaced with the same molar amount of 1,1-(2-(dimethylamino)-1,3-phenylene)bis(ethan-1-one) (compound R7-1 below), and PbO₂ (0.65 mmol) and 15 drops of concentrated HCl (12 N (concentration 37 mass %) HCl aqueous solution) were added dropwise, and except that PbO₂ (0.65 mmol) and 15 drops of HI aqueous solution (concentration 55.0 to 58.0 mass %) were used, compound 7 was obtained by the same production method as the production method for compound 1 of Example 1.

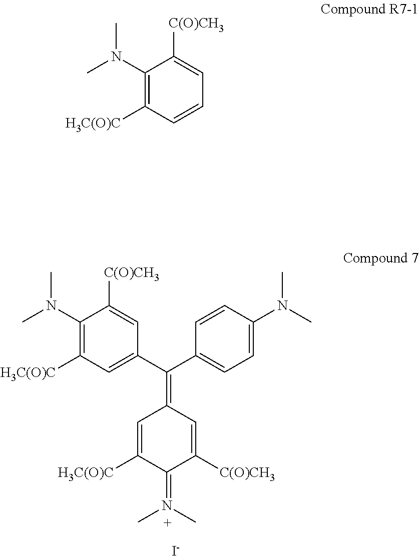

The structure of the obtained compound 7 was identified by nuclear magnetic resonance (¹H-NMR):

¹H-NMR (300 MHz, CDCl₃) σ=8.40 (s, 2H), 7.24 (s, 2H), 7.21-7.10 (m, 2H), 6.79-6.66 (m, 2H), 3.02 (s, 6H), 3.00 (s, 6H), 2.50 (s, 6H), 2.37 (s, 6H) 0.88 (s, 6H) ppm.

### (Compound 8)

Compound 8 was obtained by the same production method as in Example 1 for compound 1, except that 2-(dimethylamino)benzonitrile (40 mmol) was replaced with an equal molar amount of 3-(dimethylamino)benzene-1,2,4-tricarboxyamine (compound R8-1 below) and 4-(dimethylamino)benzaldehyde (10 mmol) was replaced with an equal molar amount of 4-(diethylamino)benzaldehyde (compound R8-2 below).

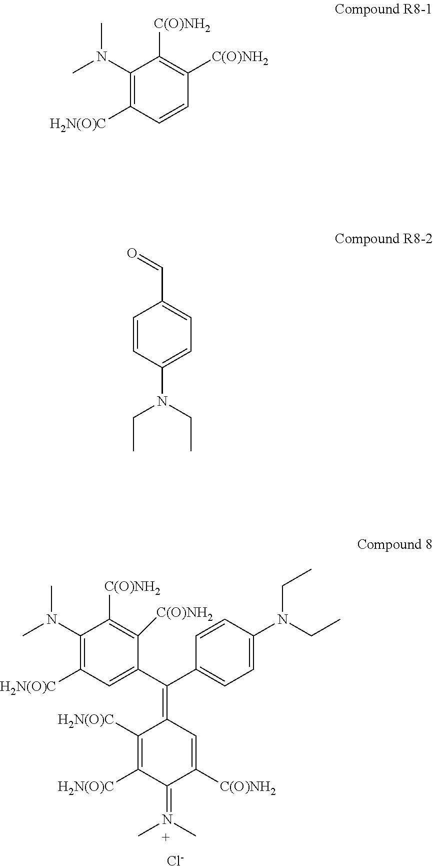

The structure of the obtained compound 8 was identified by nuclear magnetic resonance (¹H-NMR):

¹H-NMR (300 MHz, CDCl₃) σ=8.41 (s, 1H), 7.83-8.02 (m, 12H), 7.27-7.20 (m, 3H), 6.83-6.72 (m, 2H), 3.40 (q, J=7.0 Hz, 4H), 3.00 (s, 6H), 1.12 (t, J=7.1 Hz, 6H), 0.90 (s, 6H) ppm.

### (Compound 9)

Compound 9 was obtained by the same manufacturing method as the manufacturing method of compound 1 in Example 1, except that 2-(dimethylamino)benzonitrile (40 mmol) was replaced with an equal molar amount of N,N-dimethyl-2,3,5,6-tetrakis(trifluoromethoxy)aniline (compound R9-1 below).

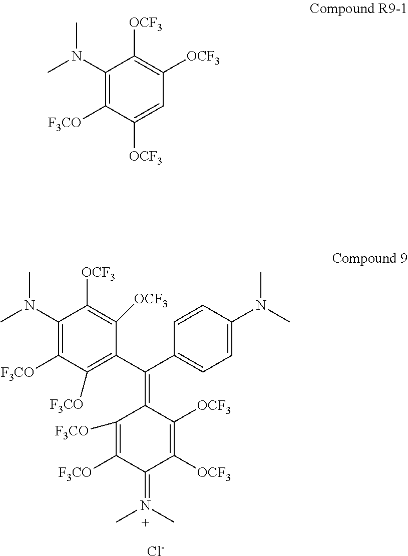

The structure of compound 9 obtained was identified by a nuclear magnetic resonance device (¹H-NMR):

¹H-NMR (300 MHz, CDCl₃) σ=7.25-7.12 (m, 2H), 6.81-6.68 (m, 2H), 3.00 (s, 6H), 2.94 (s, 6H), 0.93 (s, 6H) ppm.

### (Compound C1)

Crystal violet manufactured by Tokyo Chemical Industry Co., Ltd. was used as compound C1. Compound C1 has a structure represented by the following formula.

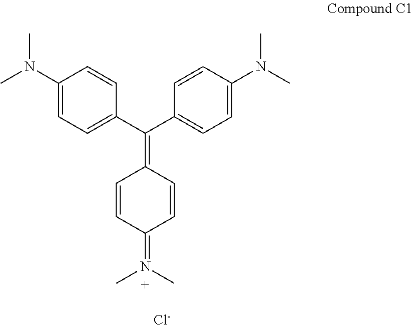

### (Compound C2)

Compound C2 was obtained by the same manufacturing method as the manufacturing method for compound 1 of Example 1, except that 2-(dimethylamino)benzonitrile (40 mmol) was replaced with the same molar amount of N,N-dimethyl-2-(trifluoromethyl)aniline (compound RC2-1 below).

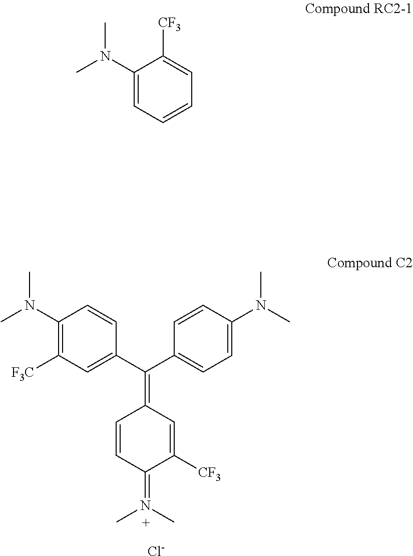

The structure of the obtained compound C2 was identified by nuclear magnetic resonance (¹H-NMR):

¹H-NMR (300 MHz, CDCl₃) σ=8.10 (s, 1H), 7.29 (s, 1H), 7.23-7.09 (m, 2H), 6.85-6.76 (m, 2H), 6.65-6.60 (m, 1H), 6.21-6.18 (m, 1H), 5.89-5.87 (m, 1H), 3.02 (s, 6H), 3.01 (s, 6H), 0.90 (s, 6H) ppm.

### <Manufacture of Electrolytic Copper Plating Solution Composition>

### (Electrolytic Copper Plating Solution Composition 1)

Each component was mixed so that the concentration of each component for 1 L of the electrolytic copper plating solution composition being manufactured was the following value, and an electrolytic copper plating solution composition 1 was manufactured. More specifically, copper sulfate, sulfuric acid, sodium chloride, and water were mixed, and the compound 1 described above, bis(3-sulfopropyl)disulfide (e.g., bis(3-sulfopropyl)persulfide, abbreviation: SPS), and polyethylene glycol were added to the obtained mixture, thereby preparing an electrolytic copper plating solution composition 1.

### (Prescription for Electrolytic Copper Plating Solution Composition 1)

Concentration: 10 mg/L of compound 1,
Concentration of copper sulfate: 100 g/L,
Concentration of sulfuric acid: 100 g/L,
Concentration of bis(3-sulfopropyl)disulfide: 5 mg/L,
Concentration of polyethylene glycol: 300 mg/L,
Concentration of sodium chloride: 50 mg/L,
water.

### (Electrolytic Copper Plating Solution Compositions 2-9, C1 and C2)

In addition, except that compound 1 was changed and compounds 2 to 9, C1 and C2 described above were used, respectively, the same manufacturing method as the manufacturing method of the electrolytic copper plating solution composition 1 described above was used to obtain electrolytic copper plating solution compositions 2 to 9, C1 and C2, respectively.

### <Evaluation>

### (Electrolytic Copper Plating)

The plating object to be plated below was acid-degreased for 3 minutes at 45° C., washed in hot water for 30 seconds at 45° C., rinsed in water for 30 seconds at 25° C., and then immersed in a concentration 10 mass % sulfuric acid aqueous solution for 1 minute at 25° C. to perform acid activation treatment.

Next, using electrolytic copper plating solution compositions 1 to 9, C1 and C2, respectively, as an electrolytic copper plating bath, the substrate and the anode (manufactured by Yamamoto Plating Tester Co., Ltd., material: titanium indium, size: 150 mm in length, 50 mm in width, and 2 mm in thickness) after acid activation treatment were immersed in the electrolytic copper plating bath, respectively connected to a power supply, and plating was performed under the following plating conditions. During the plating process, the electrolytic copper plating bath was stirred by bubbling air. Afterwards, the plated substrate was washed for 30 seconds at 25° C. and dried to manufacture each substrate having a plating film.

### <<Plating Condition>>

Negative electrode current density: 5.0 A/dm²
Plating bath: Each electrolytic copper plating solution composition obtained above
Bath temperature: 30° C.
Plating time: 30 minutes
Plating object: A substrate having a structure in which a via with a via opening diameter of 90 μm and a depth of 35 μm is formed in a resin insulating film (polyimide film) on a conductor layer (copper film), and a copper sputtered film (0.1 μm) is formed as a seed layer over the entire surface.

### (Via Conductor Recess Amount)

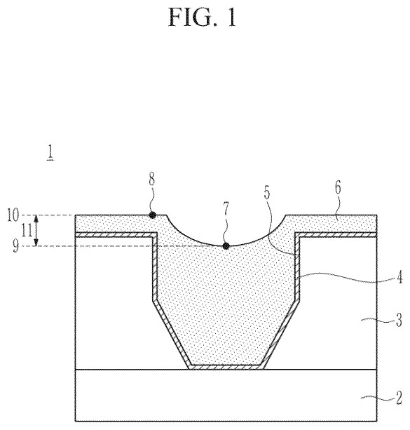

The substrate having the plating film obtained above was cut off in cross section so that the central portion of the via was included in the cross section by polishing. Using a cross-section method, which is a method of observing a cross section with a scanning electron microscope (SEM), the plating height on the periphery of the via of the plating object and the plating height at the lowest position on the bottom of the via of the plating object were measured, respectively. From the obtained measurement results, the recess amount of the via conductor (e.g., the value obtained by subtracting the plating height at the lowest point on the bottom of the via of the plating object from the plating height on the periphery of the via on the plating object) was calculated. A schematic diagram for explaining the recess amount of the via conductor is shown in FIG. 1. In FIG. 1, 1 represents a portion of a substrate having a plating film above the conductor layer. In FIG. 1, 2 represents a conductor layer, 3 represents a resin insulating film, 4 represents a seed layer, 5 represents a via, 6 represents a plating film, 7 represents a measuring point of the plating height on the via floor, 8 represents the measuring point of the via height on the perimeter, 9 represents the plating height on the via floor, 10 represents the plating height on the via perimeter, and 11 represents the recess amount of via conductor damage. The evaluation results are shown in Table 1.

### <<Evaluation Reference>>

⊚: via conductor with a recess amount of less than 2 μm,
∘: The via conductor recess amount in a range of 2 μm to 5 μm,
x: Via conductor recess amount is greater than 5 μm.

TABLE 1
Types and evaluation results of ammonium ion compounds included
in electrolytic copper plating solution compositions

| | Electrolytic Copper Plating Solution | | | |
|---|---|---|---|---|
| | Composition Used for Electrolytic | | | |
| | Copper Plating | | | |
| | | Type of | | |
| | | Ammonium | Via | |
| | | Compound in | Conductor | |
| | | Electrolytic | recess | |
| | | Copper Plating | amount | |
| | | Solution | Value | Evalu- |
| | Type | Composition | (μm) | ation |
| Embodiment 1 | Electrolytic Copper | Compound 1 | 2.8 | ◯ |
| | Plating Solution | | | |
| | Composition 1 | | | |
| Embodiment 2 | Electrolytic Copper | Compound 2 | 1.3 | ⊚ |
| | Plating Solution | | | |
| | Composition 2 | | | |
| Embodiment 3 | Electrolytic Copper | Compound 3 | 2.8 | ◯ |
| | Plating Solution | | | |
| | Composition 3 | | | |
| Embodiment 4 | Electrolytic Copper | Compound 4 | 1.9 | ⊚ |
| | Plating Solution | | | |
| | Composition 4 | | | |
| Embodiment 5 | Electrolytic Copper | Compound 5 | 1.2 | ⊚ |
| | Plating Solution | | | |
| | Composition 5 | | | |
| Embodiment 6 | Electrolytic Copper | Compound 6 | 4.9 | ◯ |
| | Plating Solution | | | |
| | Composition 6 | | | |
| Embodiment 7 | Electrolytic Copper | Compound 7 | 4.4 | ◯ |
| | Plating Solution | | | |
| | Composition 7 | | | |
| Embodiment 8 | Electrolytic Copper | Compound 8 | 3.9 | ◯ |
| | Plating Solution | | | |
| | Composition 8 | | | |
| Embodiment 9 | Electrolytic Copper | Compound 9 | 3.3 | ◯ |
| | Plating Solution | | | |
| | Composition 9 | | | |
| Comparative 1 | Electrolytic Copper | Compound C1 | 11.7 | X |
| | Plating Solution | | | |
| | Composition C1 | | | |
| Comparative 2 | Electrolytic Copper | Compound C2 | 6.6 | X |
| | Plating Solution | | | |
| | Composition C2 | | | |

From the results of Table 1, it was confirmed that the recess amount of the via conductor is reduced, the filling property of the via is excellent, and the plating film having high flatness is formed by using the ammonium salt compound of an embodiment of the present disclosure. On the other hand, in the ammonium salt compound of the comparative example, sufficient via filling properties were not obtained, and a plating film with high flatness was not obtained.

What is claimed is:

1. An ammonium salt compound represented by Chemical Formula 1: 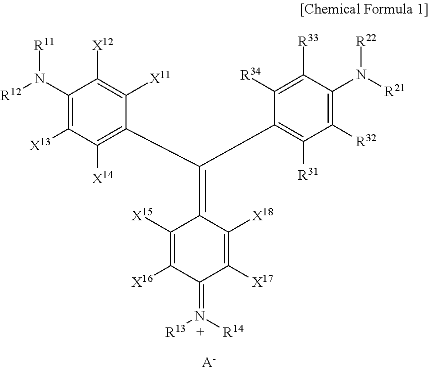
wherein in Chemical Formula 1,
R¹¹, R¹², R¹³, R¹⁴, R²¹ and R²² are each independently a methyl group or an ethyl group,
R³¹, R³², R³³ and R³⁴ are each independently a hydrogen atom, a cyano group, a nitro group, a fluoro group, a chloro group, a bromo group, an iodine group, a C₂F₅ group, an OCF₃ group, an SF₅ group, a C(O)CH₃ group, a C(O)OCH₃ group, a C(O)NH₂ group or an SO₂CH₃ group,
A is F, Cl, Br or I, and
X¹¹, X¹², X¹³, X¹⁴, X¹⁵, X¹⁶, X¹⁷, X¹⁸ are each independently a hydrogen atom, a cyano group, a C₂F₅ group, an OCF₃ group, an SF₅ group, a C(O)CH₃ group, a C(O)OCH₃ group, a C(O)NH₂ group, or an SO₂CH₃ group,
wherein at least one selected from the group consisting of X¹¹, X¹², X¹³, X¹⁴, X¹⁵, X¹⁶, X¹⁷, and X¹⁸ is not a hydrogen atom.

2. The ammonium salt compound of claim 1, wherein:
X¹¹, X¹², X¹³, X¹⁴, X¹⁵, X¹⁶, X¹⁷, and X¹⁸ are each independently a hydrogen atom, a cyano group, a C₂F₅ group, an OCF₃ group, an SF₅ group, a C(O)NH₂ group, or an SO₂CH₃ group; and
at least one selected from the group consisting of X¹¹, X¹², X¹³, X¹⁴, X¹⁵, X¹⁶, X¹⁷, and X¹⁸ is not a hydrogen atom.

3. The ammonium salt compound of claim 1, wherein A is Cl or Br.

4. The ammonium salt compound of claim 1, wherein A is Cl.

5. The ammonium salt compound of claim 1, wherein:
X¹¹, X¹², X¹³, X¹⁴, X¹⁵, X¹⁶, X¹⁷, X¹⁸ are each independently a hydrogen atom, a cyano group, a C₂F₅ group, an SF₅ group, or an SO₂CH₃ group; and
at least one selected from the group consisting of X¹¹, X¹², X¹³, X¹⁴, X¹⁵, X¹⁶, X¹⁷, and X¹⁸ is not a hydrogen atom.

6. The ammonium salt compound of claim 1, wherein:
X¹¹, X¹², X¹³, X¹⁴, X¹⁵, X¹⁶, X¹⁷, and X¹⁸ are each independently a hydrogen atom, a cyano group, a C₂F₅ group, or an SF₅ group;
at least three selected from the group consisting of X¹¹, X¹², X¹³, and X¹⁴ are not hydrogen atoms; and
at least three selected from the group consisting of X¹⁵, X¹⁶, X¹⁷, and X¹⁸ are not hydrogen atoms.

7. The ammonium salt compound of claim 1, wherein each of R₁₁, R₁₂, R₁₃, and R₁₄ is a methyl group.

8. The ammonium salt compound of claim 1, wherein each of R²¹ and R²² is a methyl group.

9. The ammonium salt compound of claim 1, wherein each of R³¹, R³², R³³, and R³⁴ is a hydrogen atom.

10. The ammonium salt compound of claim 1, wherein the ammonium salt compound comprises any one of the following Compounds 1 to 9:

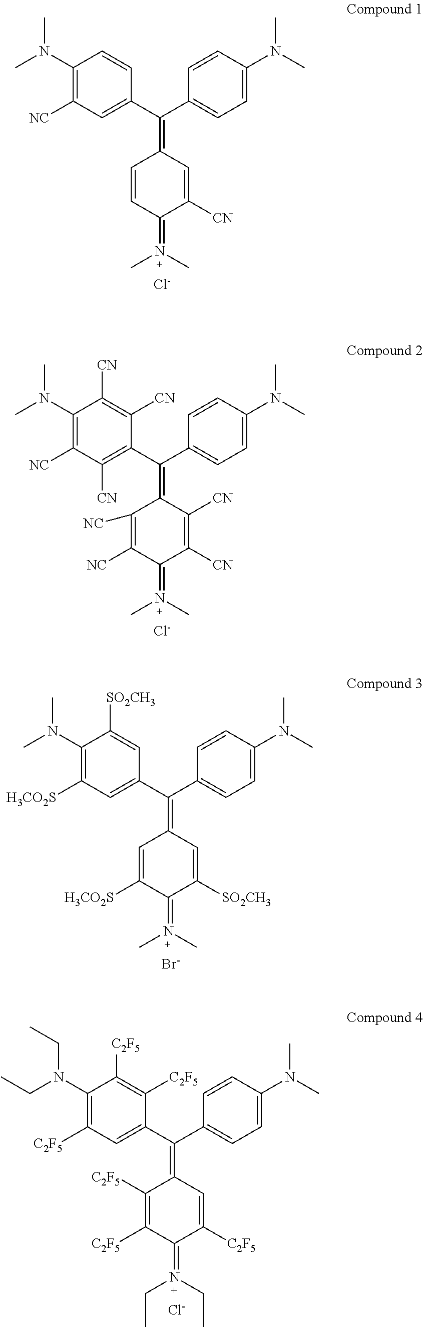

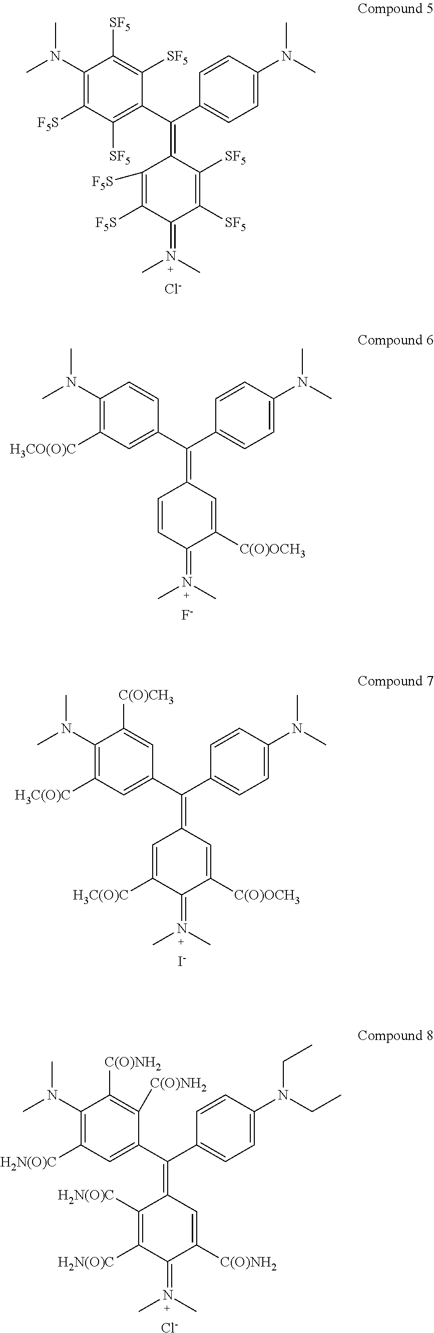

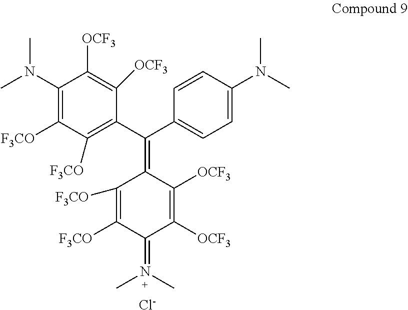

11. The ammonium salt compound of claim 1, wherein the ammonium salt compound comprises Compounds 2 or 5.

12. An additive for electrolytic copper plating consisting of the ammonium salt compound of claim 1.

13. An electrolytic copper plating solution composition comprising:
a copper ion source; and
an ammonium salt compound of claim 1.

14. The electrolytic copper plating solution composition of claim 13 further comprising at least one component selected from the group consisting of an accelerator and an inhibitor.

15. The electrolytic copper plating solution composition of claim 13 further comprising an accelerator, an inhibitor, a halide ion source, acid and water.

16. The electrolytic copper plating solution composition of claim 13, wherein the ammonium salt compound comprises at least one of the following Compounds 1 to 9:

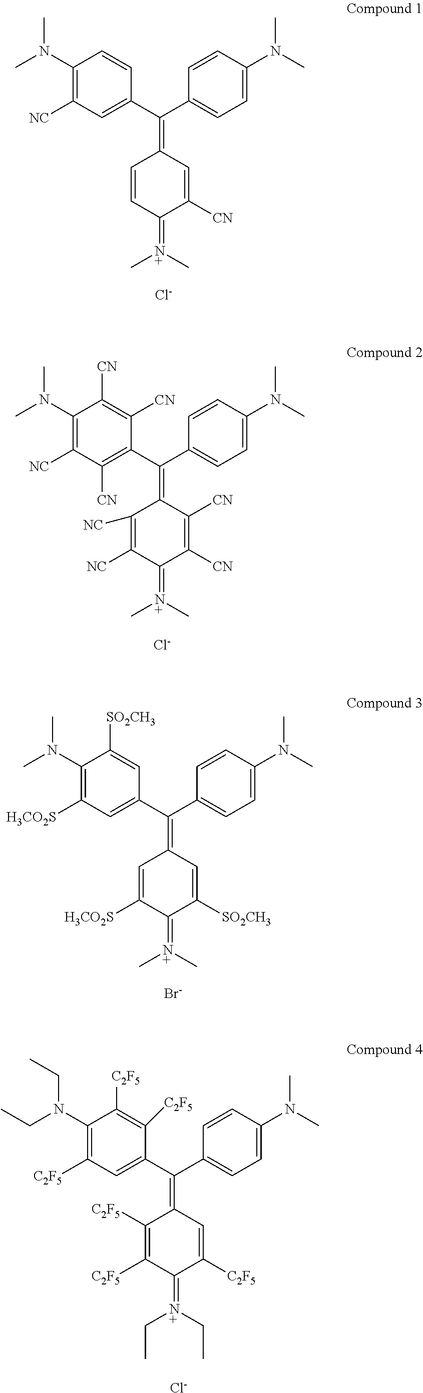

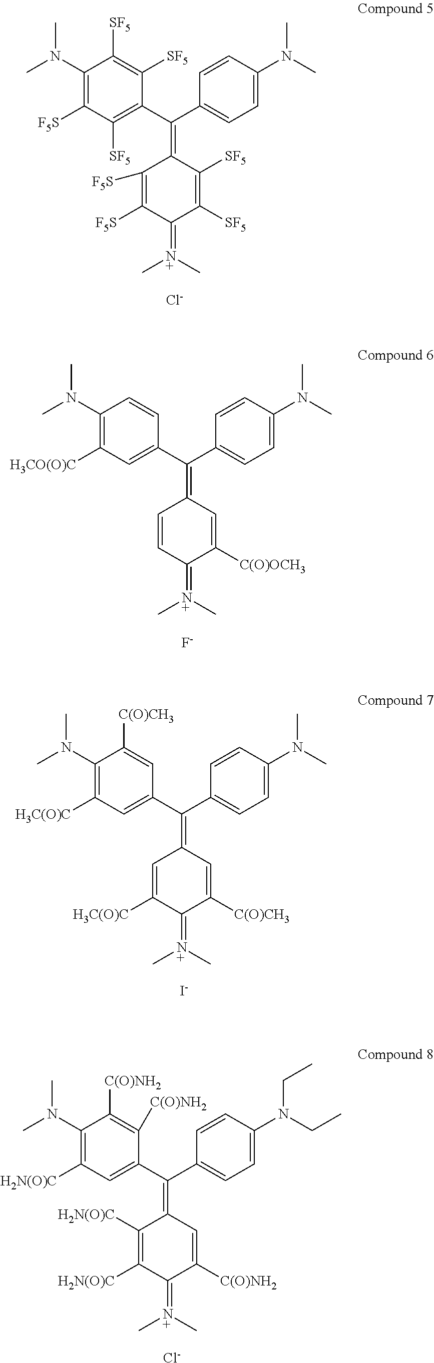

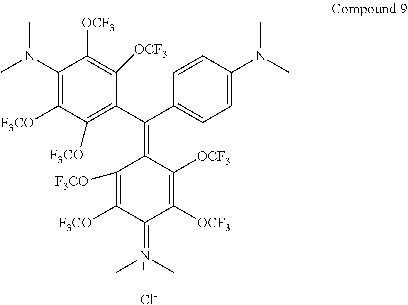

17. The electrolytic copper plating solution composition of claim 16, wherein the ammonium salt compound comprises Compounds 2 or 5.

18. The electrolytic copper plating solution composition of claim 13, wherein the ammonium salt compound comprises Compound 5.

19. An electrolytic copper plating method comprising:
forming a via conductor and a wiring pattern on a plated product using the electrolytic copper plating solution composition of claim 13.

20. An electrolytic copper plating method, comprising:
forming a via conductor and a wiring pattern on a plated product using the electrolytic copper plating solution composition of claim 14.
